(19) United States
(12) Patent Application Publication
(10) Pub. No.: US 2017/0319082 A1
(43) Pub. Date: Nov. 9, 2017

(54) Phono-Electro-Cardiogram Monitoring Unit

(71) Applicants: Siad Sayme, Hannover (DE); Corbit Inc., San Francisco, CA (US)

(72) Inventor: Siad Sayme, Hannover (DE)

(21) Appl. No.: 15/147,035

(22) Filed: May 5, 2016

(51) Int. Cl. A61B 5/0205; A61B 5/00; A61B 5/04; A61B 5/11; A61B 5/044; A61B 5/0408; H02J 7/02; A61B 7/04

(52) U.S. Cl. CPC A61B 5/02055; H02J 7/025; A61B 5/741; A61B 5/002; A61B 5/0022; A61B 7/04; A61B 5/7455; A61B 5/0006; A61B 5/1117; A61B 5/044; A61B 5/0408; A61B 5/04017; A61B 2505/07; A61B 2560/0214; A61B 2560/0431

(57) ABSTRACT

A Phono-Electro-Cardiogram Monitoring Unit that detects and monitors ECG and PCG signal comprising a phono-electro-cardiogram sensor button capable of electrical conduction of the electrical potential changes arising from the heart activity of a user or a patient. The sensor button includes a microphone which allows detection of electrocardiography signal and phonocardiogram signal simultaneously and the detected electrocardiography signal and phonocardiogram signal are processed by a multifunction microprocessor and the processed electrocardiography signal and phonocardiogram signal data are stored in a memory or be transmitted to a work station or a personal cloud for further processing and storing where the data can be forwarded to authorized individuals via wireless connectivity for communication, interpretation, service and help.

### BACKGROUND OF INVENTION

### 1. Field of the Invention

The present invention relates generally to a vital signs monitoring unit, and more particularly to an electrocardiography (ECG) and phonocardiogram (PCG) vital signs monitoring unit that is compact in size for easy carrying and use by a user or a medical staff and can be incorporated into a wearable device such that the unit can be used at any place, and at any time.

### 2. Description of Related Art

Vital signs are used to measure the body's basic functions. These measurements are taken to help assess the general physical health of a person, give clues to possible diseases, and show progress toward recovery. The normal ranges for a person's vital signs vary with age, weight, gender, and overall health. There are four primary vital signs: body temperature, blood pressure, heart rate, and respiratory rate. However, depending on the clinical setting these vital signs may include other measurements such as ECG and PCG for identifiers before providing care, treatment, or service in a clinical setting.

While vital sign monitoring has traditionally been done by doctors, a number of companies are developing portable monitoring devices which can be used by consumers themselves. One of such devices is described in US 2014/0343389 for wireless monitoring device that is suitable for attachment to the skin of a patient and where the device is capable of continuous wireless real-time measurement of physiological signals and transmission of the measurements to a computer or mobile device.

While these portable monitoring devices provides the convenience for self and remote monitoring that allows for early discovery and treatment of ailments, however, the design of these portable monitoring devices might not be compact or appeared aesthetics enough for the user when attending social events or during sporting activities. Furthermore, these portable monitoring devices still lack a more comprehensive vital sign monitoring function such as ECG and PCG, and other features that might still need to be improved upon for self-monitoring.

### SUMMARY OF THE INVENTION

The present invention overcomes the above and other drawbacks by providing a Phono-Electro-Cardiogram Monitoring Unit, in particular, the unit is capable for electrical conduction of electrical potential changes arising from the heart activity to sense and record ECG signal and simultaneously sense and record PCG signal generated during each heartbeat of a user or a patient by a microphone in the unit. As a result, the present invention increases the accuracy of various heart rate measurements, and increases the diagnostic relevance and usefulness of vital sign monitoring devices in health field, as well as for health professionals. In addition, the Phono-Electro-Cardiogram Monitoring Unit provided in the present invention can be comfortably carried and used by the user and minimizing any restriction to user's movement. Furthermore, the Phono-Electro-Cardiogram Monitoring Unit is designed in such way that it can be easily concealed, therefore, the Phono-Electro-Cardiogram Monitoring Unit can be easily blended into daily attire that can prevent the user from embarrassment or awkwardness during social events or stigmatization. Finally, the present invention provides a method for collecting, processing and monitoring ECG and PCG, and other vital sign data from the user at any place like home, hospital, caring facility, clinic, office or company and transmit the vital sign data to a secure device, which allows data access for chosen individuals like family, friends, nurses, and family and company doctors, and alert the chosen individuals when the vital data collected indicates that's the user needs immediate medical attention.

Accordingly, the present invention provides a Phono-Electro-Cardiogram Monitoring Unit for monitoring ECG and PCG of a user without hindering the user's movement and is designed to enhance the user's safety by allowing early detection of health deterioration and connecting clinicians with the user anywhere, and at any time. The Phono-Electro-Cardiogram Monitoring Unit in the present invention which comprising a phono-electro-cardiogram sensor button that is capable of detecting the ECG signal and PCG signal of the user simultaneously. The sensor button which is capable for electrical conduction of electrical potential changes arising from the heart activity of a user and simultaneously detects, stores, processes and monitors ECG signal and PCG signal generated during each heartbeat of the user. The sensor button is designed in such way that it is compact in size for easy carried by a user or be incorporated into a wearable device such that the unit can be used at any place, and at any time. In addition, the sensor button is capable of in-line or wireless communication with a work station or other external device for receiving, processing and storing the ECG and PCG signal data from the sensor button. The work station provided in the present invention comprises at least a memory hard disk integrated with a wireless module for wireless communication with the sensor button, medical algorithms, and a process interface that receives, processes, stores and forwards vital sign data from the sensor button. It is characterized that the work station sends a warning signal to the user's emergency contacts in the event that the medical algorithms result of pathologic measurements that might reflect a change in user's health condition.

Other objects and advantages of the invention herein will become apparent from the specification herein.

### BRIEF DESCRIPTION OF THE DRAWINGS

FIG. 1 is a structural view of the phono-electro-cardiogram sensor button of the present invention of Phono-Electro-Cardiogram Monitoring Unit;

FIG. 2 is an explored view of the first preferred embodiment of the phono-electro-cardiogram sensor button thereof;

FIG. 3 is an illustration of data sharing over a personal cloud;

FIG. 4 is an illustration of wireless communication between the phono-electro-cardiogram sensor button and the work station and other external devices thereof;

FIG. 5 is an embodiment view of the phono-electro-cardiogram sensor button with a wrist band thereof;

FIG. 6 is an operating illustration of the phono-electro-cardiogram sensor button thereof;

FIG. 7 is a processing illustration of the ECG signal and PCG signal in the phono-electro-cardiogram sensor button thereof;

FIG. 8 is another operating illustration of the phono-electro-cardiogram sensor button thereof;

FIG. 9 is an explored view of another preferred embodiment of the phono-electro-cardiogram sensor button thereof;

FIG. 10 is an illustration of inline connection between the phono-electro-cardiogram sensor button and the work station and a medical device thereof;

FIG. 11 is a structural view of the inductive battery recharger with the phono-electro-cardiogram sensor button thereof;

FIG. 12 is an explored view of a preferred embodiment of the inductive battery recharger thereof;

FIG. 13 is another operating illustration of the phono-electro-cardiogram sensor button;

FIG. 14 is a diagram illustrating the vital sign signal data transmission and storing steps;

FIG. 15 is an abnormal vital sign algorithm provided in the present invention; and

FIG. 16 is a diagram illustrating the method for collecting, process and monitoring vital sign signal data.

### DETAILED DESCRIPTION OF THE INVENTION

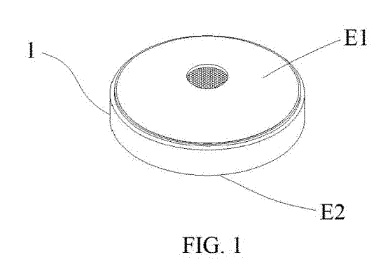

A Phono-Electro-Cardiogram Monitoring Unit that simultaneously detects, stores, processes and monitors ECG signal and PCG signal generated during each heartbeat of a user or a patient. As shown in FIG. 1, the Phono-Electro-Cardiogram Monitoring Unit comprising a phono-electro-cardiogram sensor button 1 for electrical conduction of the electrical potential changes arising from the heart activity and for detecting, storing and transmitting ECG signal and PCG signal generated during each heartbeat of the user or the patient. As a preferred embodiment, the front face of the phono-electro-cardiogram sensor button 1 is used as a first electrode E1 to be in contact with a part of user's body, and the back face of the phono-electro-cardiogram sensor button 1 is used as a second electrode E2 to be in contact with another part of user's body for detecting the continuous ECG signal of the user or the patient.

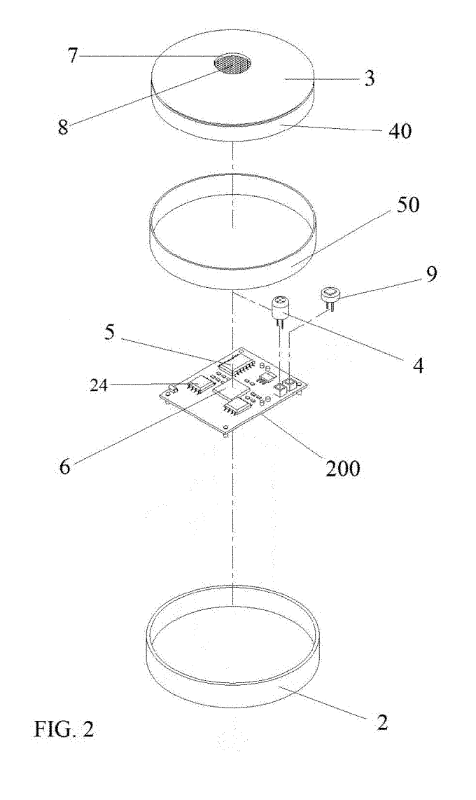

As shown in FIG. 2, the phono-electro-cardiogram sensor button 1 comprising an electrical conductive button body 2 as the first electrode E1 to be used in contact with a part of user's body and an electrical conductive panel 3 as the second electrode E2 to be used in contact with another part of the user or the patient's body for detecting the continuous ECG signal of the user. It should be noted that any material that is capable of electrical conduction can be used for the electrical conductive button body 2 and the electrical conductive panel 3.

As shown in FIG. 1, the electrical conductive button body 2 and the electrical conductive panel 3 forms a circular phono-electro-cardiogram sensor button 1 that houses various components of the phono-electro-cardiogram sensor button 1 described hereafter. However, any variation of shape of the phono-electro-cardiogram sensor button 1 can be adapted, such as square, oval or any other geometric shapes.

To prevent electric conduction between the electrical conductive button body 2 and the electrical conductive panel 3, an insulating medium is provided in between the electrical conductive button body 2 and the electrical conductive panel 3 for electrical isolation. As shown in FIG. 2, as an insulating medium, the bottom edge 40 or the portion of the electrical conductive panel 3 that inserted into the electrical conductive button body 2 is made of electricity insulating material to prevent electrical conduction between the electrical conductive button body 2 and the electrical conductive panel 3. In another embodiment, as shown in FIG. 2, an insulating ring 50 is provided in the electrical conductive button body 2 to prevent electrical conduction between the electrical conductive button body 2 and the electrical conductive panel 3.

As shown in FIG. 2, the phono-electro-cardiogram sensor button 1 further comprising a circuit board 200 provided in the electrical conductive button body 2 and covered by the electrical conductive panel 3. The phono-electro-cardiogram sensor button 1 further comprising a microphone 4 for detecting PCG signal during each heartbeat of the user, a memory 5 for storing ECG signal and PCG signal data, and at least one multifunction microprocessor 6 that electrically connected to the electrical conductive button body 2 and the electrical conductive panel 3 for processing the detected vital sign signal and controlling various components of the sensor button 1, all of which, are mounted on the circuit board 200.

As a preferred embodiment, the microphone 4 is provided with a sensitivity larger than 7.9 mV/Pa±2 dB at 1 kHz, and a frequency response between 20 Hz to 600 Hz and a S/N ratio of larger than 58 dB for optimal internal heart beat listening.

As shown in FIG. 2, as a preferred embodiment, an opening 7 is provided on the electrical conductive panel 3 to allow the PCG signal reaches the microphone 4. As a preferred embodiment, the opening 7 is covered by a membrane 8 to allow amplification for specific frequencies of PCG signal generated by each heartbeat of the user or the patient to be detected by microphone 4. The membrane 8 is also useful when the sensor button 1 is placed in other part of user's body rather than the chest to detect other internal organ sound. In this setting, the membrane 8 would allow the sensor button 1 to detect both low and high audio frequencies for cross reference of the diagnosis.

As shown in FIG. 2, as a preferred embodiment, the sensor button 1 is provided with a LED light 9 mounted on the circuit board 200 for various operation feedbacks and indicating the area of contact for electrical conduction panel 3 on the user or the patient's body. The LED light 9 may glow through the opening 7 or through the membrane 8 on the electric conductive panel 3, or a separate opening for the LED light 9 may be provided on the electric conductive panel 3. Additional LED light may also be added to the sensor button 1 for various indications.

As shown in FIG. 2, as a preferred embodiment, the phono-electro-cardiogram sensor button 1 further comprising a first wireless module 24 mounted on the circuit board 200 for wireless communicating and transmitting the ECG signal and PCG signal data detected by the phono-electro-cardiogram sensor button 1 to a work station for further processing and storing and for communication with other external devices. The first wireless module 24 may adapt Wi-Fi, 4G, Bluetooth or other similar communication method.

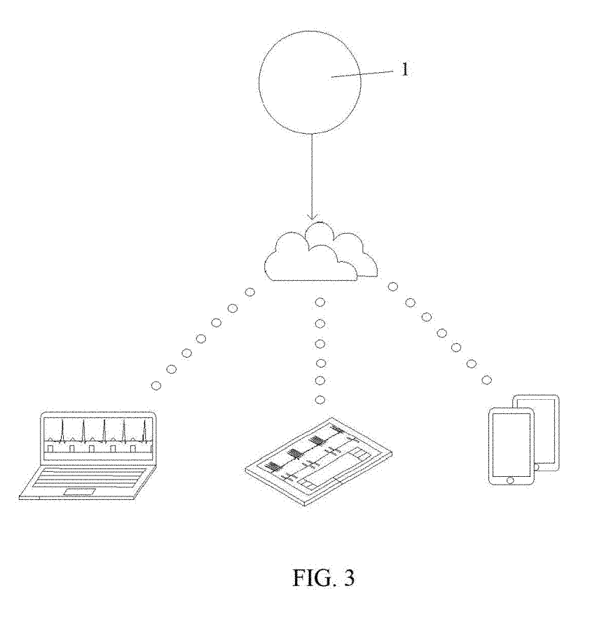

As shown in FIG. 3, as a preferred embodiment, the first wireless module may also be used for uploading ECG signal and PCG signal data in the phono-electro-cardiogram sensor button 1 to a personal cloud for storing and sharing with authorized individuals. The authorized individuals may download the ECG signal and PCG signal data of the user via a cellphone, tablet computer or other handheld electronic devices.

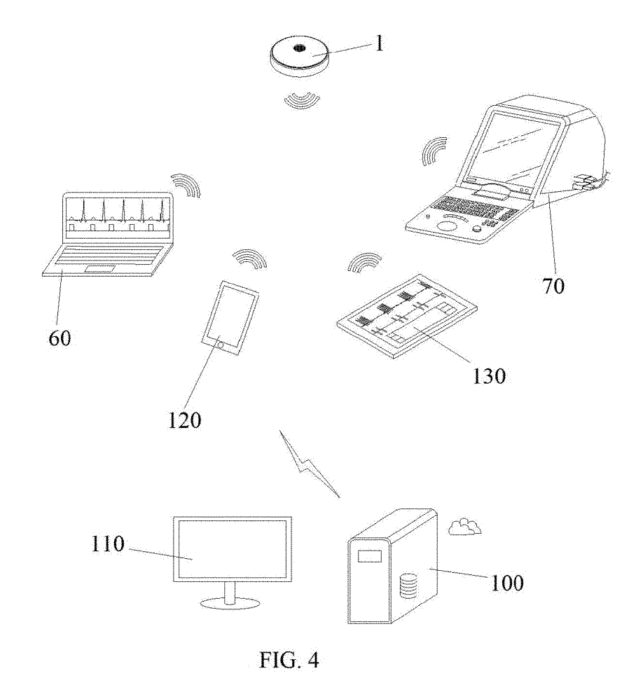

As shown in FIG. 4, as a preferred embodiment, the work station can be placed in a physical location such as home or office of the user. The work station in this embodiment can be a computer 100 which comprising at least a memory hard disk integrated with a second wireless module for wireless communication with the first wireless communication module 24 in the phono-electro-cardiogram sensor button 1 and other authorized devices, medical algorithms, a process interface that receives, processes, stores and forwards vital sign data received from the phono-electro-cardiogram sensor button 1 to other authorized external devices via the second wireless module and a display module that displays graphic representation of the vital sign data received and allowing videoconferencing over the display module. In one embodiment, the display module can be a computer monitor 110 or any other type of display screen. The second wireless module may adapt Wi-Fi, 4G, Bluetooth or other similar communication method. In another embodiment, the work station can also be in the form of a laptop computer 60 or a handheld personal electronic device to be carried by the user or medical staff, such as a cellphone 120 or a tablet computer 130. In addition, as another preferred embodiment, as shown in FIG. 3, the phono-electro-cardiogram sensor button 1 can be adapted via the first wireless module 24 to wirelessly connect to other external devices such as a medical device 70 for a more comprehensive medical diagnosis. The medical device 70 in this instance may include heart assist device like LVAD or artificial heart and an implantable pacemaker, defibrillator, as well as implantable devices for heart insufficiency like CRT (Cardiac Resynchronization Therapy) or CCM (Cardiac Contractility Modulation).

In one embodiment, a communication interface (not shown in the drawing) can be provided for the phono-electro-cardiogram monitoring unit to enable a remote communication and access of the vital sign data store in the phono-electro-cardiogram sensor button and the work station by the user, or by the authorized emergency contacts and the medical service center wirelessly via an external device. This communication interface can be a computer software or a downloadable App.

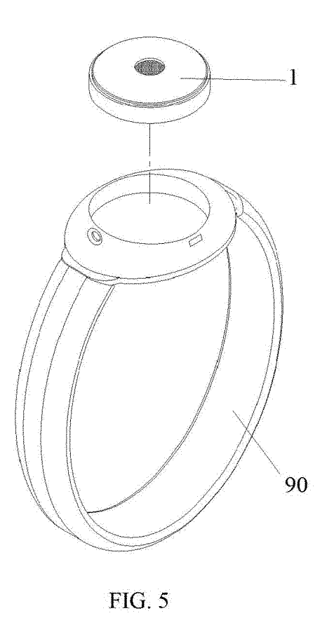

As a preferred embodiment, the phono-electro-cardiogram sensor button 1 can be combined with a wearable device or a hand held device or being designed to be used itself as a wearable or portable device or be a portable device able to be transformed to a wearable by getting connected to a bracelet, necklace or belt or be inserted into a clothing. As a preferred embodiment, as shown in FIG. 5, the phono-electro-cardiogram sensor button 1 is provided in a matching shape to be inserted into a matching cavity provided on an outer surface of a wrist band 90. As a preferred embodiment, the wrist band 90 is made of soft flexible, waterproof and antibacterial material for comfortable fitting and easy cleaning.

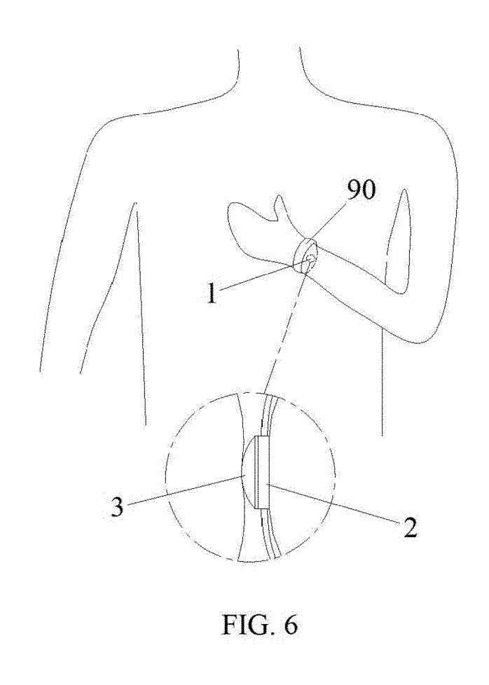

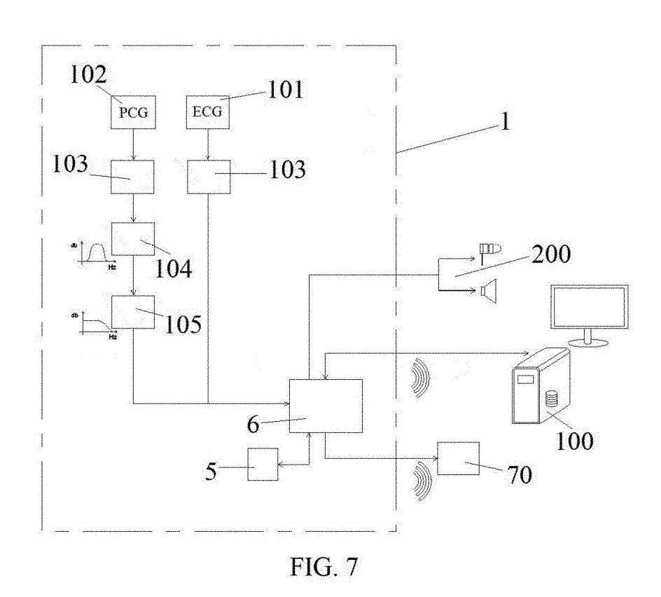

When using the phono-electro-cardiogram sensor button 1 to detect ECG and PCG signal, as shown in FIG. 6, for which the phono-electro-cardiogram sensor button 1 is incorporated with a wrist band 90, the user would place the electrical conductive panel 3 against the chest, and at the same time, the bottom surface of the electrical conductive button body 2 would be pushed against the user's wrist to complete an electrode cycle for ECG reading. During this operation, the microphone 4 in the phono-electro-cardiogram sensor button 1 also detects the user's PCG signal. As shown in FIG. 7, once the ECG signal 101 and the PCG 102 signal are detected by the sensor button 1, the ECG signal 101 and the PCG signal 102 are first amplified by an amplifier circuit 103 provided in the phono-electro-cardiogram sensor button 1. The amplified ECG signal 101 is then processed by the multifunction microprocessor 6. Such process by the multifunction microprocessor 6 would include processing the ECG signal 101 from analog signal into digital signal. Once processed, the ECG signal 101 is then stored in the memory 5 or be wirelessly transmitted to a work station 100, such as a computer 100 or other external device such as the medical device 70 for further processing and storing. The amplified PCG signal 102 is then filtered by a bandpass filter circuit 104 mounted on the circuit board 200 in the phono-electro-cardiogram sensor button 1. The bandpass filter circuit 104 is set at PCG: 20˜600 Hz for optimal wireless transmission or output of PCG signal 102 at a later time. Once filtered, the PCG signal 102 is then denoised by a noise reduction chip 105 provided in the phono-electro-cardiogram sensor button 1. Once denoised, the PCG signal 102 can then be output to an audio device 200, such as a speaker or an earphone, or is then processed by the multifunction microprocessor 6. Such process by the multifunction microprocessor 6 would include processing the PCG signal 102 from analog signal into digital signal. Once processed, the PCG signal 102 is then stored in the memory 5 or be wirelessly transmitted to the work station 100 or other external device such as the medical device 70 for further processing and storing.

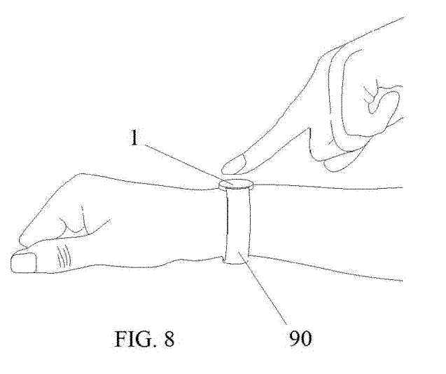

In another usage of the phono-electro-cardiogram sensor button 1, to do a quick ECG reading, as shown in FIG. 8, for which the phono-electro-cardiogram sensor button 1 is incorporated with a wrist band 90, the user would simply touch the electrical conductive panel 3 with one finger of the other hand to complete an electrode cycle for ECG reading.

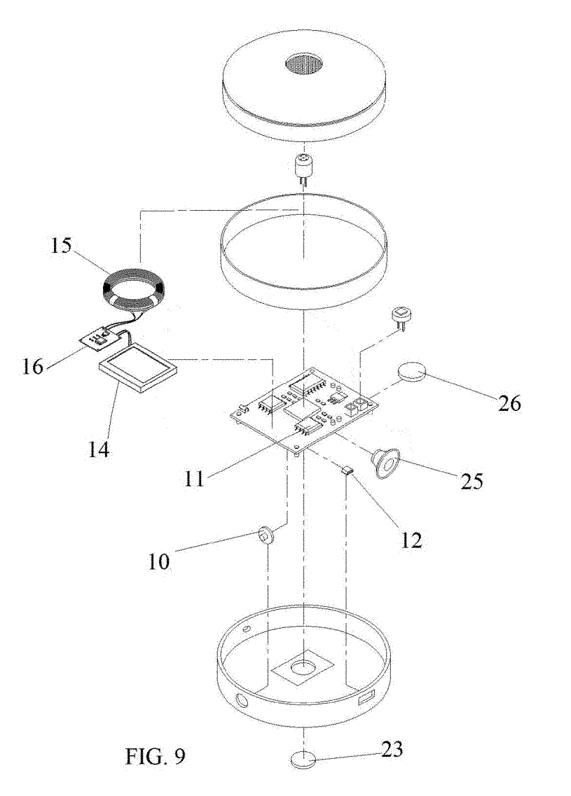

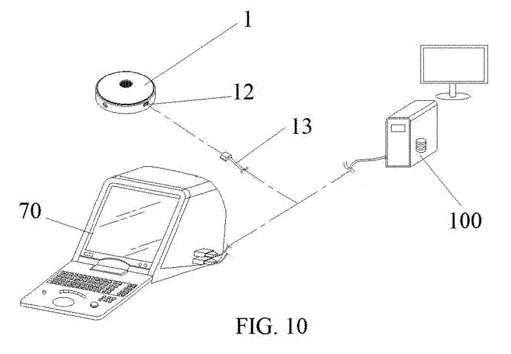

As shown in FIG. 9 and FIG. 10, in one embodiment, the phono-electro-cardiogram sensor button 1 is provided with a connecting port 12 mounted on the circuit board 200 and a connecting line 13 for connecting the phono-electro-cardiogram sensor button 1 to a work station 100, an external power source or other external devices. The work station 100 can be a computer. The other external device can be a handheld personal electronic device or a medical device 70, such as a blood pressure monitor (Sphygmomanometer), a blood sugar monitor, a blood-clotting monitor, an ECG recorder or an EEG recorder, an ECG Recording devices, like a Holter Monitors and Loop or Event Recorders, a Wearable Cardioverter Defibrillator (WCD), a patient Monitoring systems for ambulatory or intensive care, ambulatory Blood pressure monitoring (ABPM), an echo machine, a stethoscope, a patient Monitoring systems for ambulatory or intensive care, a Heart-Catheter Laboratory, an Intracardial Ultrasound, an IVUS (Intravascular Ultrasound) or Optical Coherence Tomography (OCT) and a heart pressure measurement by heart catheter (CVC, Right and left heart catheter, FFR and iFR) and solutions for cardiac output measurements (PiCCO). The connecting port 12 may be adapted a micro USB port or other similar port and the connecting line 13 may be adapted a universal connecting cable.

As shown in FIG. 9, in one embodiment, the phono-electro-cardiogram sensor button 1 is provided with an on/off switch 10 and a power control chip 11 mounted on the circuit board 200 for operating requests and turning off the phono-electro-cardiogram sensor button 1 when not using the unit to prolong operation time. The phono-electro-cardiogram sensor button 1 can be turned on or off by pressing and holding the switch 10 for two seconds. Once the phono-electro-cardiogram sensor button is turned on, various operation functions can be access by a quick press and release the switch 10. As an added function, if no vital sign signal is received for a time period of 30 seconds, the power control chip 11 triggers the phono-electro-cardiogram sensor button 1 into a hibernating mode to further minimize power consumption.

As shown in FIG. 9, in one embodiment, the phono-electro-cardiogram sensor button 1 is provided with a rechargeable battery 14 mounted on the circuit board 200 for providing an internal power to the phono-electro-cardiogram sensor button 1. A rechargeable battery 14 can provide the needed power to run the sensor button 1 around the clock. A typical rechargeable battery can provide enough power to run the sensor button 24 hours a day for a number of days before the need to be recharged.

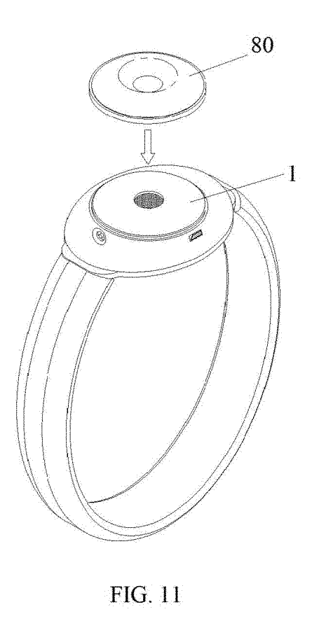

As shown in FIG. 9 and FIG. 11, as a preferred embodiment, to allow for uninterrupted vital sign monitoring and to save the trouble and time for replacing the battery, the rechargeable battery 14 is provided with a first inductive charging coil unit for wireless battery charging with an inductive battery charger 80 for attaching to the phono-electro-cardiogram sensor button 1. The first inductive battery charging coil unit comprising a charging coil 15 and a microchip 16 for which are placed in the electrical conductive button body 2 and are covered by the electrical conductive panel 3.

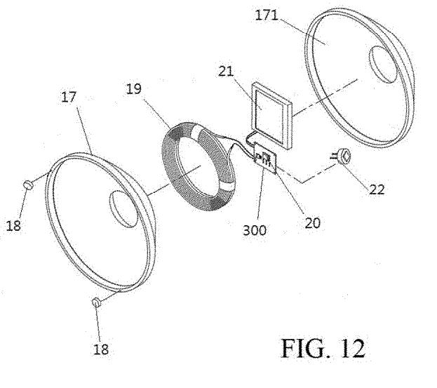

As shown in FIG. 12, as a preferred embodiment, the inductive battery charger 80 provided in the present invention comprising an electrical conductive panel matching charger casing 17 and a top casing 171 that forms a hollow inductive battery charger body, with at least one magnet 18 provided at bottom side of the charger casing 17 for attaching to the surface of the electrical conductive panel 3. The charger casing 17 which houses a second inductive charging coil unit which comprising an inductive charging coil 19, a circuit board 300, a microchip 20, a battery 21 and a LED back light 22. The inductive charging coil 19 and battery 21 are electrically connected to the circuit board 300, and the microchip 20 and the LED back light 22 are mounted on the circuit board 300. When recharging the inductive battery charger 80, the inductive battery charger 80 can be placed at any wireless charging station. Once fully charged, the LED back light 22 on the inductive battery charger 80 will flash rapidly. As shown in FIG. 5, when the power in the battery of the sensor button 1 is low, which can be indicated by a rapid flash of the LED light 9 on the sensor button 1, the user can simply attach the inductive battery charger 80 to the surface of the electrical conductive panel 3, for which, the magnets 18 provided on the inductive battery charger 80 will adhere to any metal surface on the electrical conductive panel 3. Once adhered, the second inductive charging coil unit in the battery charger 80 will create an electromagnetic field and induces a current in the first inductive charging coil unit and start the charging process of the rechargeable battery 14. Once the rechargeable battery 14 is fully charged, which can be indicated by the dimming of the LED light 9 on the sensor button 1, the user can simply reattach the inductive battery charger 80.

As shown in FIG. 9, as a preferred embodiment, the phono-electro-cardiogram sensor button 1 further comprising at least one physiological sensor 23 placed on the bottom of the electrical conductive button body 2. The physiological sensor 23 can be provided in, but electrically isolated from electrical conductive button body 2 and is covered by the electrical conductive panel 3. The physiological sensor 23 can be used to measure the user's vital sign which can include heart rate, pulse rate, body temperature, Pulse Oximetry, Respiration Rate, Electrodermal Activity (EDA), Electroencephalography (EEG), Electromyography (EMG), Electroneurography (ENG) and combinations thereof.

As shown in FIG. 9, in one embodiment, the phono-electro-cardiogram sensor button 1 further comprising a speaker 25 for voice communication with emergency contacts or medical center and for playing operation, advice and warning messages. An emergency contact or medical service center can be connected to the first wireless module 24 in the sensor button 1 via an external device, such as a cell phone or a computer, to have a wireless conversation with the user on the speaker 25. Depending on the setting, the speaker 25 can also be set to play various operating and warning messages, as well as the heart sound of the user when using the sensor button 1.

As shown in FIG. 9, in one embodiment, the phono-electro-cardiogram sensor button 1 further comprising a vibration mechanism 26 mounted on the circuit board 200 for additional operation feedbacks. The vibration mechanism 26 may vibrate to alert the user of various operation and warning conditions. The vibration mechanism 26 can also be turned off depending on user's setting.

As a preferred embodiment, the phono-electro-cardiogram sensor button 1 can wirelessly via the first wireless module 24, or via the connecting cable 13, connect to the work station 100 for setting various default functions of the phono-electro-cardiogram sensor button 1, for manually transferring vital sign data form the memory 5 to the work station 100, or for setting up contact list by entering contact's phone numbers.

As a preferred embodiment, the medical algorithms provided in the work station 100 constantly compare the vital sign data received from the phono-electro-cardiogram sensor button 1 for any pathologic measurements that might reflect a change in user's health condition. In the event a pathologic measurement is determined, the work station 100 sends a warning signal to the phono-electro-cardiogram sensor button 1 to alert the user. Depending on the criticalness of the pathologic measurement, the work station 100 can send an alert signal to the phono-electro-cardiogram sensor button 1 to trigger the LED light 9 to flash, the vibration mechanism 26 to vibrate or to send a voice message to be played via the speaker 25. This alert signal can be sent repeatedly in a preset time interval until the situation has been taken care of. If a more dire pathological measurement is determined, the work station can also send an alert signal to the emergency contacts and medical service center for immediate situation control.

In one embodiment, the phono-electro-cardiogram sensor button 1 maybe also provided with medical algorithms for comparing the vital sign data within the sensor button 1. In the event a pathologic measurement is determined, the sensor button 1 triggers a warning signal to alert the user.

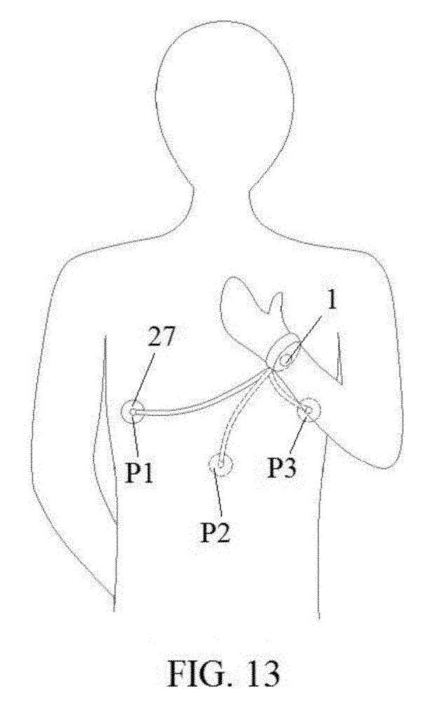

In another embodiment, as shown in FIG. 13, the Phono-Electro-Cardiogram Monitoring Unit may comprise at least one independent electrode conductive patch 27 as a third electrode to be attached to a part of the user's body. The electrode conductive patch 27 may be connected to the phono-electro-cardiogram sensor button 1 by a connecting line. This connecting line may be permanently fixed onto the phono-electro-cardiogram sensor button 1 or by plug-in. The electrode conductive patch 27 may be placed at right chest apex P1, at left chest apex P3 or at epigastrium P2. Other placement of the third electrode conductive patch 27 may be in accordance with augmented unipolar limb lead such as I, II, III (Einthoven leads), aVL, aVF and aVR (Goldberger leads) and in case of positioning all three additional electrodes can virtually calculate the 6 classical left Chest leads (Wilson leads), as well as right chest leads, to more accurately detect a right heart attack. Additional electrode conductive patch can be added to the unit by design. In this preferred embodiment, the second and the third electrode may be used interchangeably with the first electrode for detecting ECG signal of the user.

The present invention also provides a method for detecting, processing, storing and monitoring PCG signal and PCG signal, and at least one other vital sign of a user comprises a wearable vital sign monitor, such as the phono-electro-cardiogram sensor button 1 described herein, that detects, stores and transmits ECG signal and PCG signal data and at least one other vital sign data group consisting of heart rate, pulse rate, body temperature, blood pressure, respiration rate and combinations thereof. The method also comprises a work station for receiving, processing, storing and transmitting the vital sign data received from the wearable vital sign monitor to other external devices. The work station can be a computer 100 described herein to placed in any physical location such as user's home or office. And finally, the method also including a personal cloud to securely store and share important vital sign data of a user.

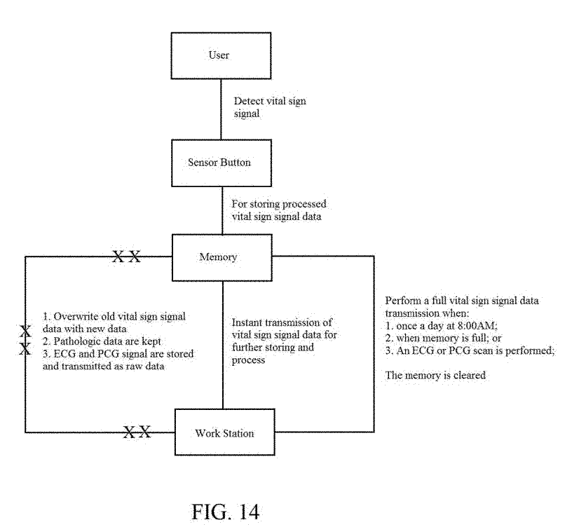

As shown in FIG. 14, in order to monitor vital sign data and maintain important vital sign data, the method delegates the wearable vital sign monitor to perform a full vital sign data transmission wirelessly to the work station for storing and processing and to the personal cloud for secure storing and sharing in the following events:

(1) once a day at a predetermine time, such as 03:00 AM;
(2) when the wearable vital sign monitor storage is full; and
(3) when the wearable vital sign monitor performs an ECG or PCG scan.

Once the data is fully transmitted to the work station and the personal cloud all content in the wearable vital sign monitor memory is then cleared to make room for subsequent vital sign data storing.

In addition, the method also delegates the wearable vital sign monitor to responds to a measurement request sent from the work station and triggers a full data transmission.

In the event that no connection to the work station and the personal cloud can be established and the memory in the wearable vital sign monitor is full, the method delegates the wearable vital sign monitor to perform as least one of the following actions to make room in the memory for any new vital sign data:
(1) keeping new data as priority compared to older measurements: cycle storage: erase older data; and
(2) keep pathologic data as priority before new measurements: don't overwrite important data; and
(c) by user's selection, the ECG signal and PCG signal detected are immediately transmitted as raw data to the work station for processing and storing to save memory storage.

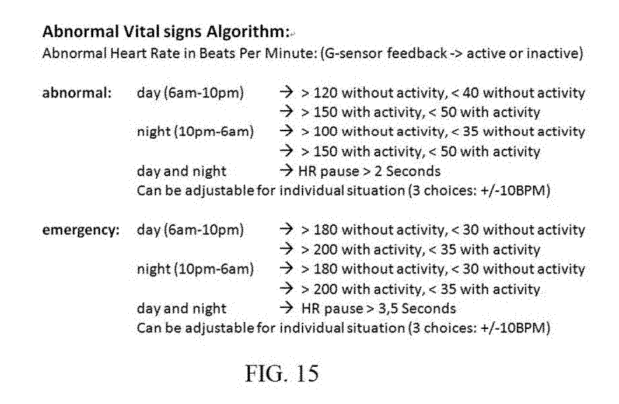

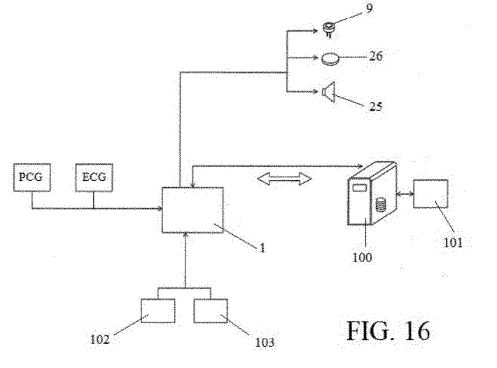

As shown in FIG. 15 and FIG. 16, the work station 100 is provided with algorithms 101 for calculating the abnormal vital signs, such as ECG and PCG signal, or any other above-mentioned vital sign signal received from the phono-electro-cardiogram sensor button 1. An algorithm for abnormal heart beats per minute is shown in FIG. 14. As shown in FIG. 15, in the event of abnormal ECG and PCG activity or abnormal other vital signs detected by the algorithm in the work station, such as heart beats per minute, the work station triggers the the phono-electro-cardiogram sensor button 1 to perform as least one of the following action to alert the user:

(1) a light-emitting diode light status indicator 9 provided in the wearable vital sign monitor flashes;
(2) a vibration mechanism 26 provided in the phono-electro-cardiogram sensor button 1; and
(3) a voice warning message with a recommended course of action to be taken is played on a speaker 25 in the phono-electro-cardiogram sensor button 1.

In the event that any of the above action is triggered the vital sign data is immediately transmitted to the work station and the personal cloud, and the data is marked as abnormal. This step is repeated every 15 seconds until a manual cancellation of monitoring by the user.

As shown in FIG. 16, in the event of emergency heart rate activity detected by the algorithm 101 in work station 100, such as heart beats per minute, the work station triggers the phono-electro-cardiogram sensor button 1 to perform as least one of the following actions:

(1) the light-emitting diode light status indicator 9 provided in the phono-electro-cardiogram sensor button 1;
(2) the vibration mechanism 26 provided in said wearable vital sign monitor vibrates; and
(3) a voice warning message with a recommended course of action to be taken is played on a speaker 25 in the phono-electro-cardiogram sensor button 1.

In the event any of the above action is triggered the vital sign data is immediately transmitted to the work station 100 and the personal cloud, and the data is marked as emergency.

In the event that no manual cancellation of the monitoring by the user is recorded within two minutes from the first instance of the data that is marked as emergency, an alert from the work station is sent to designated emergency contacts or medical service center for immediate attention.

The method also allows user to configure the work station to set three levels of attention modes for vital sign data transmitting to designated emergency contacts or medical service center:
(a) First level: Daily Healthcare Mode: a default mode, the data is transmitted to the work station and the personal cloud at a daily basis at a predetermined time;
(b) Second level: Watch Me Mode: the data is transmitted to the work station and the personal cloud once every 15 minutes; and
(c) Third level: Hospital Mode: the data is sent to the work station and the personal cloud once every minute.

In addition, a designated emergency contact or medical service center is allowed to request the most recent data from the work station via a hand held device or a computer. Such request triggers a new measurement request to the wearable vital sign monitor and the new data is transmitted and processed in the work station and then transmitted to the requested designated emergency contact or medical service center's hand held device or computer.

And finally, as shown in FIG. 16, the method also incorporates a gravity sensor 102 and a proximity sensor 103 along with the microphone in the wearable vital sign monitor for fall down detection of the user and increasing accuracy to avoid false alarm by additionally sensing sudden change in heart rate or artefact and impact sound.

Although the invention has been explained in relation to its preferred embodiment, it is to be understood that many other possible modifications and variations can be made without departing from the spirit and scope of the invention as hereinafter claimed.

1. A Phono-Electro-Cardiogram Monitoring Unit comprising a phono-electro-cardiogram sensor button for electrical conduction of the electrical potential changes arising from the heart activity; wherein said phono-electro-cardiogram sensor button comprising an electrical conductive button body as a first electrode to be used in contact with a part of a user or a patient's body and an electrical conductive panel as a second electrode to be used in contact with another part of the user or the patient's body for detecting the continuous electrocardiography signal of the user; wherein, an insulating ring is provided in said electrical conductive button body for electrical isolation between said electrical conductive button body and electrical conductive panel; wherein said phono-electro-cardiogram sensor button further comprising a circuit board provided in said electrical conductive button body and covered by said electrical conductive panel; wherein a microphone for detecting phonocardiogram signal during each heartbeat of the user, a memory, at least one multifunction microprocessor all of which are mounted on said circuit board; wherein, said electrical conductive button body and electrical conductive panel are electronically connected to said multifunction microprocessor; wherein, an opening is provided on said electrical conductive panel to allow the heart sound signal reaches said microphone; and wherein, said phono-electro-cardiogram sensor button detects electrocardiography signal and phonocardiogram signal simultaneously and said detected electrocardiography signal and phonocardiogram signal are processed by said multifunction microprocessor and the processed electrocardiography signal and phonocardiogram signal data are stored in said memory.

2. The Phono-Electro-Cardiogram Monitoring Unit as claimed in claim 1 wherein, said microphone has a sensitivity larger than 7.9 mV/Pa±2 dB at 1 kHz, and a frequency response between 20 Hz to 600 Hz and a S/N ratio of larger than 58 dB for the best result but not necessary limited to this range.

3. The Phono-Electro-Cardiogram Monitoring Unit as claimed in claim 1 wherein, said opening is covered by a membrane to allow amplification for specific frequencies of signal generated by each heartbeat of the user to be detected by said phono-electro-cardiogram sensor button.

4. The Phono-Electro-Cardiogram Monitoring Unit as claimed in claim 1 wherein, said phono-electro-cardiogram sensor button is provided in a matching shape to be inserted into a matching cavity provided on an outer surface of a wearable device or a hand held device.

5. The Phono-Electro-Cardiogram Monitoring Unit as claimed in claim 1 wherein, said phono-electro-cardiogram sensor button further comprising at least one physiological sensor mounted on said circuit board; and wherein, said physiological sensor measures the user's vital sign is selected from a group consisting of heart rate, pulse rate, body temperature, blood pressure, respiration rate and combinations thereof.

6. The Phono-Electro-Cardiogram Monitoring Unit as claimed in claim 1 wherein, said phono-electro-cardiogram sensor button further comprising a connecting port mounted on said circuit board for connecting said phono-electro-cardiogram sensor button to a work station or an external device; wherein, said connecting port is provided with a cable line for connecting said phono-electro-cardiogram sensor button to said work station or external device; and wherein, said external device is a computer, a handheld personal electronic device, a medical device, or a power source.

7. The Phono-Electro-Cardiogram Monitoring Unit as claimed in claim 1 wherein, said phono-electro-cardiogram sensor button further comprising a first wireless module mounted on said circuit board for wireless communication and transmitting the electrocardiography signal and phonocardiogram signal data detected by said phono-electro-cardiogram sensor button to a work station, a personal cloud or an external device for processing, storing and sharing data; and wherein, said external device is a computer, a handheld personal electronic device, or a medical device.

8. The Phono-Electro-Cardiogram Monitoring Unit as claimed in claim 7 wherein, said phono-electro-cardiogram sensor button further comprising a bandpass filter circuit mounted on said circuit board; and wherein, said bandpass filter circuit is set at 20˜600 Hz for optimal wireless transmission of the electrocardiography signal and phonocardiogram signal to said work station or other external devices.

9. The Phono-Electro-Cardiogram Monitoring Unit as claimed in claim 1 wherein, said phono-electro-cardiogram sensor button further comprising a rechargeable battery mounted on said circuit board for providing internal power to said phono-electro-cardiogram sensor button.

10. The Phono-Electro-Cardiogram Monitoring Unit as claimed in claim 9 wherein, said rechargeable battery is provided with a first inductive charging coil unit for wireless battery charging with an inductive battery charger; and wherein, said first inductive charging coil unit comprising a charging coil and a microchip.

11. The Phono-Electro-Cardiogram Monitoring Unit as claimed in claim 10 wherein, said inductive battery charger comprising an electrical conductive panel matching charger casing and a top casing with at least one magnet provided at bottom side of said charger casing for attaching to said electrical conductive panel; and wherein, said charger casing which houses a second inductive charging coil unit; and said second inductive charging coil unit comprising an inductive charging coil, a circuit board, a microchip, a battery and a light-emitting diode back light.

12. The Phono-Electro-Cardiogram Monitoring Unit as claimed in claim 1 wherein, said phono-electro-cardiogram sensor button further comprising an on/off switch and a power control chip mounted on said circuit board for operating requests and turning off the unit or said switching phono-electro-cardiogram sensor button to hibernating mode when not using to prolong operation time.

13. The Phono-Electro-Cardiogram Monitoring Unit as claimed in claim 1 wherein, said phono-electro-cardiogram sensor button further comprising a speaker mounted on said circuit board for voice communication with emergency contacts or medical service center, and for playing operation, advice and warning messages.

14. The Phono-Electro-Cardiogram Monitoring Unit as claimed in claim 1 wherein, said phono-electro-cardiogram sensor button further comprising a light-emitting diode light mounted on said circuit board for operation feedback and indicating the area for electrical conduction panel of the said phono-electro-cardiogram sensor button on the user's body.

15. The Phono-Electro-Cardiogram Monitoring Unit as claimed in claim 1 wherein, said phono-electro-cardiogram sensor button further comprising a vibration mechanism mounted on said circuit board for operation feedbacks.

16. The Phono-Electro-Cardiogram Monitoring Unit as claimed in claim 6 wherein, said work station is to be placed in a physical location such as home or office of the user or in the form of a hand held device to be carried by the user or medical staff; it is characterized that said work station comprising at least a memory hard disk integrated with a second wireless module for wireless communication with the first wireless communication module in said phono-electro-cardiogram unit and other authorized external devices, medical algorithms, a process interface that receives, processes, stores and forwards vital sign data from said phono-electro-cardiogram sensor button to other authorized external devices via the second wireless module and a display module that displays graphic representation of the vital sign data received and allowing videoconferencing with the user over the display module; and it is characterized that said work station sends a warning signal to emergency contacts or medical service center in the event that said medical algorithms determines a pathologic measurement that might reflect a change in user's health condition.

17. The Phono-Electro-Cardiogram Monitoring Unit as claimed in claim 1 wherein, said phono-electro-cardiogram sensor button further comprising at least one independent electrode conductive patch as a third electrode to be attached to a part of the user or the patient's body; and wherein, said electrode conductive patch is connected to said phono-electro-cardiogram sensor button by a connecting line.

18. A method for detecting, processing, storing and monitoring electrocardiography signal and phonocardiogram signal, and at least one other vital sign of a user or a patient comprising:
a phono-electro-cardiogram monitoring sensor button that detects, stores and transmits electrocardiography signal and phonocardiogram signal, and at least one other vital sign data group consisting of heart rate, pulse rate, body temperature, respiration rate and combinations thereof; wherein,
a work station that receives, stores, processes and transmits vital sign data received from said phono-electro-cardiogram monitoring sensor button;
a personal cloud that receives, stores and shares vital sign data received from said phono-electro-cardiogram monitoring sensor button; wherein,
said phono-electro-cardiogram monitoring sensor button performs a full vital sign data transmission wirelessly to the work station and the personal cloud in the following events:
(1) once a day at a predetermine time;
(2) when said phono-electro-cardiogram monitoring sensor button memory is full; and
(3) when said phono-electro-cardiogram monitoring sensor button performs a electrocardiography or phonocardiogram scan; wherein,
once the data is fully transmitted to said work station and the personal cloud all content in a memory in said phono-electro-cardiogram monitoring sensor button is then cleared to make room for subsequent vital sign data storing; wherein,
said phono-electro-cardiogram monitoring sensor button also responds to a measurement request sent from said work station and triggers a full data transmission; and wherein, in the event no connection to said work station or the personal cloud is established and the storage is full said phono-electro-cardiogram monitoring sensor button performs as least one of the following actions:
(1) keeping new data as priority compared to older measurements: cycle storage: erase older data; and
(2) keep pathologic data as priority before new measurements: don't overwrite important data; and
(3) by selection, the electrocardiography signal and phonocardiogram signal are raw data transmitted to said work station and personal cloud for processing and storing to save memory storage.

19. The method for collecting, processing, storing and monitoring vital sign of a user as claimed in claim 18 wherein, in the event of abnormal electrocardiography signal and phonocardiogram signal activity or abnormal other vital signs detected by the work station, the work station triggers said phono-electro-cardiogram monitoring sensor button to perform as least one of the following actions to alert the user:
(1) a light-emitting diode light status indicator provided on said phono-electro-cardiogram monitoring sensor button flashes;
(2) a vibration mechanism provided in said phono-electro-cardiogram monitoring sensor button vibrates; and;
(3) a voice warning message with a recommended course of action to be taken is played on said phono-electro-cardiogram monitoring sensor button; wherein, in the event that any of the above action is triggered the vital sign data is immediately transmitted to said work station and the data is marked as abnormal; and this step is repeated every 15 seconds until a manual cancellation of said phono-electro-cardiogram monitoring sensor button by the user.

20. The method for collecting, processing, storing and monitoring vital sign of a user as claimed in claim 18 wherein, in the event of emergency heart rate activity detected by the work station, said phono-electro-cardiogram monitoring sensor button performs as least one of the following actions:
(1) a light-emitting diode light status indicator provided on said phono-electro-cardiogram monitoring sensor button flashes;
(2) a vibration mechanism provided in said phono-electro-cardiogram monitoring sensor button vibrates; and;
(3) a voice warning message with a recommended course of action to be taken is played on said phono-electro-cardiogram monitoring sensor button; wherein,
in the event any of the above action is triggered the vital sign data is immediately transmitted to the work station and the data is marked as emergency; and in the event that no manual cancellation of the monitoring by the user is recorded within two minutes from the first instance of the data is marked as emergency, an alert from the work station is sent to designated emergency contacts, medical service center or emergency department.

21. The method for collecting, processing, storing and monitoring vital sign of a user as claimed in claim 18 wherein, said work station is configured to allow the user to set three levels of attention modes for data transmitting to designated emergency contacts or medical service center:
(a) First level: Daily Healthcare Mode: a default mode, the data is transmitted to the work station and the personal cloud at a daily basis at a predetermined time;
(b) Second level: Watch Me Mode: the data is transmitted to the work station and the personal cloud once every 15 minutes; and
(c) Third level: Hospital Mode: the data is sent to the work station and the personal cloud once every minute; and wherein,
a designated emergency contact or medical service center is allowed to request the most recent data from said work station via hand held device or a computer, such request triggers a new measurement request to said phono-electro-cardiogram monitoring sensor button and the new data is transmitted and processed in said work station and then transmitted to the requested designated emergency contact or medical service center's hand held device or computer.

22. The method for collecting, processing, storing and monitoring vital sign of a user as claimed in claim 18 wherein, said wearable vital sign monitor incorporates a gravity sensor and a proximity sensor with a microphone in said phono-electro-cardiogram monitoring sensor button for fall down detection of the user and increasing accuracy to avoid false alarm by additionally sensing sudden change in heart rate or artefact and impact sound.
